(19) United States
(12) Patent Application Publication
(10) Pub. No.: US 2021/0244266 A1
(43) Pub. Date: Aug. 12, 2021

(54) TOOLS AND METHODS FOR CLEANING LUMENS OF MEDICAL DEVICES

(71) Applicant: Jacek KRZYZANOWSKI, Etobicoke (CA)

(72) Inventor: Jacek KRZYZANOWSKI, Etobicoke (CA)

(21) Appl. No.: 17/167,269

(22) Filed: Feb. 4, 2021

Related U.S. Application Data
(60) Provisional application No. 62/971,396, filed on Feb. 7, 2020.

(51) Int. Cl. A61B 1/12; B08B 9/043; A46B 1/00; A46B 15/00; B08B 9/057; A46B 3/18; A46B 9/02

(52) U.S. Cl. CPC A61B 1/122; B08B 9/0436; A46B 1/00; A46B 15/0081; A46B 2200/3013; A46B 3/18; A46B 9/026; B08B 2209/04; B08B 9/057

(57) ABSTRACT

Cleaning tools and associated methods for cleaning lumens of medical devices are provided. An embodiment of the cleaning tool comprises an elongated core, two scrubbers attached to the elongated core, and a wiper attached to the elongated core. The wiper is axially disposed between the two scrubbers. The cleaning tool may be used to clean the lumen prior to decontamination of the lumen.

### CROSS REFERENCE TO RELATED APPLICATION AND CLAIM OF PRIORITY

The present application claims priority to U.S. provisional patent application No. 62/971,396 filed on Feb. 7, 2020, the entire contents of which are hereby incorporated herein by reference.

### TECHNICAL FIELD

The disclosure relates generally to cleaning medical devices, and more particularly to tools and methods for cleaning medical devices.

### BACKGROUND

It is paramount that reusable medical devices be cleaned prior to high disinfection or sterilization. The current standard published as Technical Information Report (TIR) 30:2011 by the Association for the Advancement of Medical Instrumentation (AAMI) requires less than 6.4μg/cm² of residual surface protein level after the cleaning process in order to allow the subsequent disinfection/sterilization process to achieve the proper sterility assurance level.

Many medical devices contain lumens through which surgical tools can be passed, tissue removed, liquids and/or gasses delivered or evacuated, implants placed, etc. The lumens are difficult to clean as they are not easily accessible. Also, endoscope channels and any other lumens in reusable medical/surgical devices may have surface imperfections such as scratches and small voids caused by regular use and which can make them difficult to clean. Improvement is desirable.

### SUMMARY

In one aspect, the disclosure describes a lumen cleaning tool for cleaning a lumen of a medical device. The lumen cleaning tool comprises:

an elongated core having a longitudinal axis;

a first bristle brush attached to the elongated core;

a second bristle brush attached to the elongated core, the first and second bristle brushes being axially spaced apart along the longitudinal axis of the elongated core; and

one or more disc-shaped squeegees attached to the elongated core and disposed axially between the first and second bristle brushes.

In another aspect, the disclosure describes a lumen cleaning tool for cleaning a lumen of a medical device. The lumen cleaning tool comprises: an elongated core; two scrubbers attached to the elongated core and spaced apart along the elongated core; and a wiper attached to the elongated core and disposed between the two scrubbers.

In a further aspect, the disclosure describes a method of cleaning a lumen of a medical device. The method comprises:

propelling a cleaning tool in the lumen along a direction of movement; and while propelling the cleaning tool in the lumen along the direction of movement, using the cleaning tool to, in a single pass of the cleaning tool in the lumen:

scrub a wall defining the lumen;

after scrubbing the wall, wipe the wall; and

after wiping the wall, scrub the wall.

In a further aspect, the disclosure describes a method of propelling a lumen cleaning tool including a scrubber and a wiper along a lumen of a medical device. The method comprises, when the lumen cleaning tool is disposed inside the lumen, using a pressure differential along the lumen to propel the lumen cleaning tool along the lumen.

Further details of these and other aspects of the subject matter of this application will be apparent from the detailed description included below and the drawings.

### DESCRIPTION OF THE DRAWINGS

Reference is now made to the accompanying drawings, in which:

FIG. 1 is a perspective view of an exemplary lumen cleaning tool for cleaning lumens of medical devices;

FIG. 2 is a side elevation view of the tool of FIG. 1;

FIG. 3 is a side elevation view of the tool of FIG. 1 disposed inside a lumen of a medical device and being propelled inside the lumen;

FIG. 4 is a side elevation view of another exemplary lumen cleaning tool disposed inside a lumen of a medical device and being propelled inside the lumen;

FIG. 5 is a side elevation view of a scrubber of the cleaning tool of FIG. 4;

FIG. 5A is a magnified view of bristles of the scrubber of FIG. 5;

FIG. 6 is a side elevation view of another exemplary lumen cleaning tool;

FIG. 7 is a flow diagram of a method for cleaning a lumen of a medical device;

FIG. 8 is a flow diagram of a method of propelling a lumen cleaning tool along a lumen of a medical device using a pressure differential; and

FIG. 9 is a side view of the lumen cleaning tool of FIG. 1 disposed inside a lumen of a medical device and propelled along the lumen using a pressure differential.

### DETAILED DESCRIPTION

The following disclosure describes tools and associated methods for cleaning lumens of reusable medical (e.g., surgical) devices (e.g., endoscopes) that subsequently have to be decontaminated (e.g., disinfected and/or sterilized) between patient uses. In some embodiments, the tools and methods described herein may reduce the likelihood of residual contaminants such as biofilms being left on lumen walls after cleaning. In some embodiments, the tools and methods described herein may also reduce the likelihood of contaminants remaining trapped in scratches, voids or other surface defects that may be found on lumen walls. In some embodiments, the tools and methods described herein may promote cleaning of medical devices to a level facilitating subsequent decontamination.

Aspects of various embodiments are described through reference to the drawings.

The terms “attached” or “secured” may include both direct attachment/securement (in which two elements contact each other) and indirect attachment/securement (in which at least one additional element is located between the two elements).

The term “substantially” as used herein may be applied to modify any quantitative representation which could permissibly vary without resulting in a change in the basic function to which it is related.

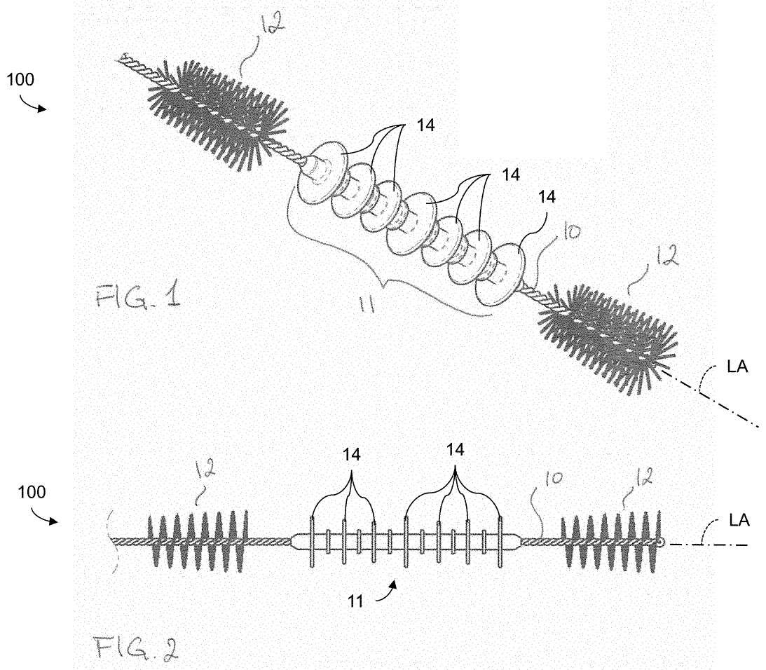

FIGS. 1 and 2 are respective perspective and side elevation views of an exemplary lumen cleaning tool 100 (referred hereinafter as “tool 100”). Tool 100 may comprise elongated core 10, one or more wipers 11 and one or more scrubbers 12. Core 10 may have a longitudinal axis LA and may have an axial length that is greater than that of the portion of core 10 shown in FIG. 1. Wiper 11 and scrubbers 12 may be secured to core 10. Wiper 11 may be located axially between two scrubbers 12 along the length of core 10. Core 10 may include a wire, twisted wires, a tube, a rod, a catheter, or a combination thereof. Core 10 may be of a unitary construction or may include multiple elements (e.g., axial sections) assembled together. Core 10 may be flexible, semi-rigid or substantially rigid along its entire axial length. Core 10 may have a non-uniform flexibility that varies along its length. As an example, core 10 may include a flexible section followed by a rigid section. An exemplary core 10 shown in FIG. 1 includes a pair of twisted (e.g., metallic, stainless steel) wires.

Core 10 may be of any length and thickness (e.g., diameter) that suit the size and configuration of the lumen to be cleaned, and that accommodate the desired number and configuration of wipers 11 and scrubbers 12 included in tool 100. In some non-limiting embodiments, core 10 may have a thickness (e.g., diameter) that is between 0.3 mm and 2.0 mm and may have a length that is between 50 mm and 2400 mm.

Wipers 11 and scrubbers 12 may be attached to and positioned along core 10 of cleaning tool 100. Wipers 11 and scrubbers 12 may be sized to allow contact (i.e., frictional engagement) with the lumen's wall(s) for mechanically removing the contaminants (e.g., debris, solids, liquids) inside of the lumen. In other words, the outer diameters of wipers 11 and scrubbers 12 may be larger than the diameter of the lumen to provide an interference fit within the lumen as shown in FIGS. 3, 4 and 9.

Wiper 11 may include one or more (e.g., a set) flexible disc-shaped squeegees 14 as shown in FIG. 1 that may be made of a shape-conforming thermoplastic. For example, squeegees 14 may be made of a suitable thermoplastic elastomer (TPE), thermoplastic rubber (TPR), thermoplastic polyurethane (TPU) and/or thermoplastic vulcanizate (TPV). Squeegees 14 may be flexible radially-extending fins that are axially distributed along core 10. Alternatively or in addition, wiper 11 may include one or more 0-rings made of silicon or any other suitable material, one or more sponges made of a moisture-expanding material, and/or one or more inflatable balloons configured to maintains close contact with the wall(s) defining the lumen. Wiper 11 may be continuous along its length along longitudinal axis LA of core 10. Alternatively, wiper 11 may include a plurality of axial sections separated by axial gaps exposing core 10. In various embodiments, wiper 11 may be attached to core 10 by suitable means (e.g., adhesive) or may be overmolded onto core 10 using injection molding for example.

The dimensions of wiper 11 may be of any length and thickness that suit the size of the lumen to be cleaned. In some non-limiting embodiments, wiper 11 may have a thickness (e.g., diameter) that is between 1 and 15 mm and a length that is between 20 and 100 mm.

Scrubbers 12 may include twist-in wire (e.g., bristle) brushes secured to core 10 as shown in FIG. 1. Scrubbers 12 may include twisted wire brushes including bristles that are held in place by, and which extend radially from, twisted wire core 10. The bristles may also be circumferentially distributed around core 10. To form the twisted wire brush, the bristles may be inserted between parallel wires and then the wires may be twisted together to press and retain the bristles therebetween. Other suitable methods of securing bristles to core 10 of different types may be used. Depending on the intended application, the density of the bristles and the surface area covered by the bristles may be varied by adjusting the number of bristles and/or by angling the bristles at desired angles from longitudinal axis LA.

The bristles may be made of suitable materials (e.g., polymer, nylon) having suitable physical dimensions, flexibility, and other characteristics according to the intended application. In general, the dimensions of the scrubbers 12 can be of any length and thickness that suit the size of the lumen to be cleaned. In some non-limiting embodiments, the scrubbers 12 may have a thickness (e.g., diameter) that is between 1 and 15 mm and a length that is between 20 and 100 mm. Scrubbers 12 may be continuous along their length along longitudinal axis LA of core 10. Alternatively, one or more both scrubbers 12 may include a plurality of axial sections separated by axial gaps exposing the core 10.

Alternatively or in addition, scrubbers 12 may include one or more abrasive sponges, molded brushes, abrasive fibers, or any other elements containing a scrubbing media. Scrubbers 12 may be attached to and positioned along core 10 of tool 100 and may be sized to allow contact between the scrubbers 12 and the wall(s) defining the lumen.

Tool 100 may also incorporate other features (not shown), such as size limiters preventing tool 100 entry into (e.g., undersized) lumens of predetermined sizes or shapes, and/or leaders allowing easy entry of tool 100 into lumens.

FIG. 3 is a side view of tool 100 disposed inside lumen L of a medical device and being propelled inside lumen L in direction D. FIG. 3 shows an axial cross-sectional view of lumen L to expose tool 100 disposed therein. Lumen L may be a channel of a flexible or rigid endoscope for example. During operation, tool 100 may be propelled inside lumen L either manually, semi-automatically or automatically so that wiper 11 and scrubbers 12 may cooperatively expel the contaminants out of lumen L. For example, tool 100 may be manually pushed or pulled through lumen L by manually pushing or pulling on core 10. Alternatively or in addition, tool 100 may be propelled through lumen L by way of an actuator drivingly coupled to tool 100, and/or by way of a pressure differential (e.g., positive or negative pressure) axially across wiper 11 as explained further below for example.

Tool 100 may include wiper 11 that is disposed axially between front scrubber 12F and back scrubber 12B in relation to direction D of movement relative to lumen L. As tool 100 is moved axially inside lumen L in direction D by pushing or pulling tool 100 along the lumen L, front scrubber 12F is disposed ahead of wiper 11 and scrubs and loosens the contaminants from the wall(s) defining lumen L. The wiper 11 then wipes (e.g., squeegees) the contaminants and moves them forward in the direction D of the axial movement of tool 100 and eventually expels them out of lumen L. However, the frictional engagement of wiper 11 with the wall(s) defining lumen L may, in some situations, cause a relatively thin biofilm to be left behind on the wall(s) of lumen L. Such biofilm could be difficult to remove in one or more subsequent decontamination steps. Furthermore, the wall(s) defining lumen L may not be perfectly smooth. For example, lumen L may have surface imperfections such as scratches and voids. Wiper 11 may push some contaminants into these surface imperfections, making them even more difficult to remove in subsequent decontamination steps such as rinsing/flushing with detergent-based solutions.

In some situations, the use of back scrubber 12B being propelled behind wiper 11 along direction D may facilitate subsequent decontamination steps by scrubbing and loosening the residual biofilm or other contaminants that may be left behind wiper 11. Such scrubbing and loosening of the contaminants behind wiper 11 may enhance the efficiency of the subsequent rinsing/flushing steps that may be involved in cleaning or decontaminating lumen L. Accordingly, the combination of scrubbers 12F, 12B and wiper 11 disposed axially therebetween may cause a series of cooperating steps to be carried out in a sequence that enhances cleaning, and that may also facilitate subsequent decontamination of lumen L in some situations.

In some embodiments, scrubbers 12F, 12B may have substantially identical constructions. For example, scrubbers 12F, 12B may have a same density of bristles, and may also have bristles of the same cross-sectional thickness (e.g., diameter) and length. Alternatively, scrubbers 12F, 12B may have different constructions as explained below.

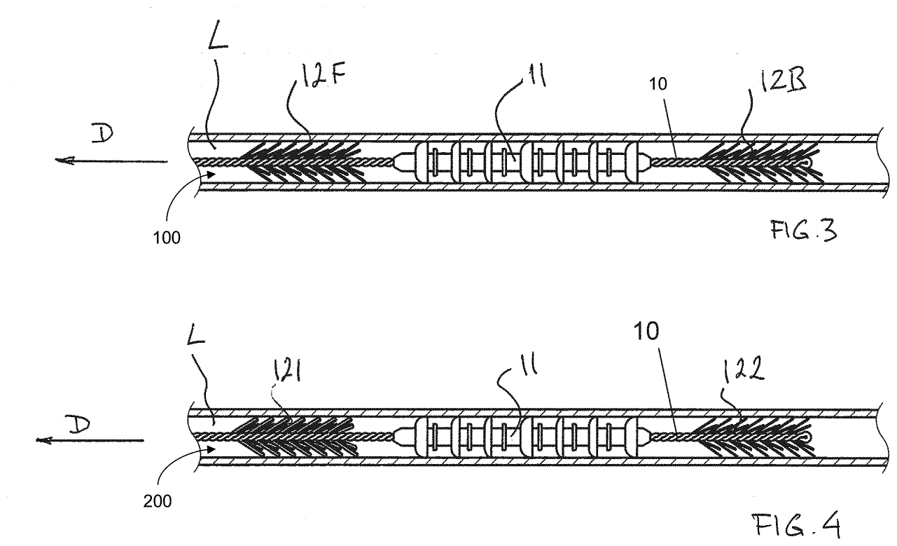

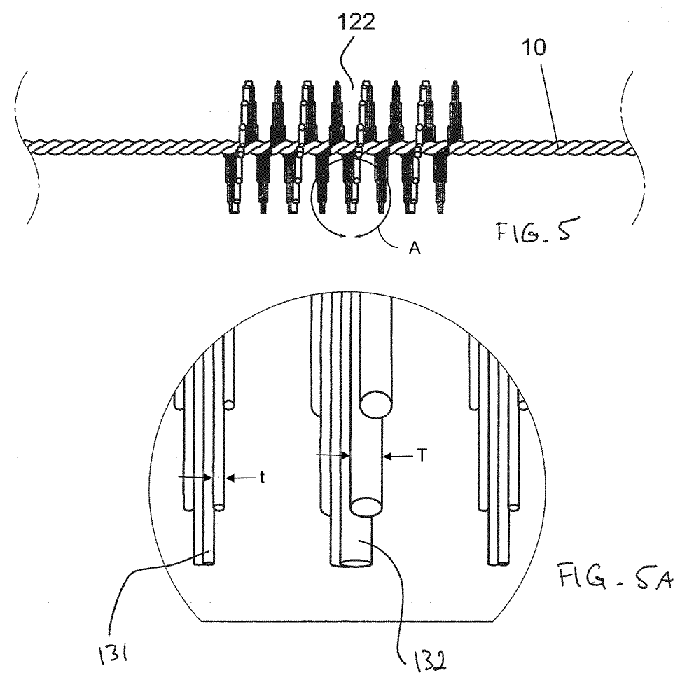

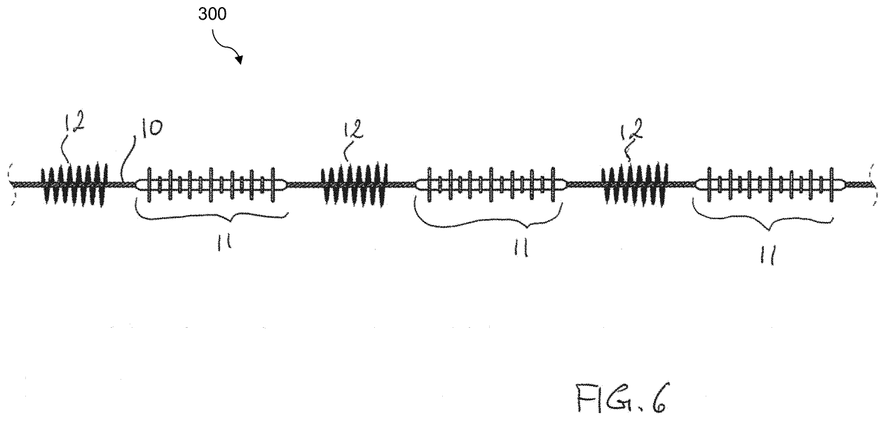

FIG. 4 is a side view of another exemplary lumen cleaning tool 200 (referred hereinafter as “tool 200”) disposed inside lumen L of a medical device and being propelled inside lumen L along direction D. FIG. 4 shows an axial cross-sectional view of lumen L to expose tool 200 disposed therein. Tool 200 may include core 10, scrubbers 121, 122 and wiper 11 disposed axially between scrubbers 121, 122. Scrubbers 121, 122 may be bristle brushes. In contrast with tool 100, scrubbers 121 and 122 of tool 200 may have different constructions from each other. For example, the bristles in front scrubber 121 and back scrubber 122 may have different cross-sectional thicknesses (e.g., diameters). For example, back scrubber 122 may have bristles of smaller thickness than those of front scrubber 121. For example, back scrubber 122 may be a relatively fine bristle brush and front scrubber 121 may include a relatively coarse bristle brush.

Front scrubber 121 may have (i.e., coarser) bristles of a larger thickness that, during operation, more aggressively scrub and loosen the contaminants from the wall(s) defining lumen L ahead of wiper 11. On the other hand, back scrubber 122 may have (i.e., finer) bristles of a smaller thickness that may interact more desirably with the thin scratches and small voids in the lumen's wall. Accordingly, the bristles of smaller thickness of back scrubber 122 may be better adapted to scrub and release contaminants from relatively small surface imperfections in the lumen's wall(s) and may facilitate subsequent cleaning or decontamination steps.

FIG. 5 is a side view of an exemplary back scrubber 122 of tool 200 shown of FIG. 4. FIG. 5A is a magnified view of bristles 131, 132 of back scrubber 122. In some embodiments, back scrubber 122 may include a combination of bristles 131 of smaller thickness t and bristles 132 of larger thickness T that may cooperate together to provide desired scrubbing action on the wall(s) defining lumen L. The arrangement of bristles 131, 132 in back brush 122 can be such that sections of thinner bristles 131 and thicker bristles 132 are alternatively arranged along core 10. Alternatively, thinner bristles 131 and thicker bristles 132 may be randomly arranged or otherwise mixed within back scrubber 122. Back scrubber 122 may contain bristles of two or more (e.g., three, four or five) different thicknesses. The cross-sectional profile(s) of bristles 131, 132 may be circular, oval, rectangular or any other suitable shape. Bristles 131, 132 may be solid or hollow.

During operation, thicker bristles 132 of back scrubber 122 may more aggressively scrub and loosen the biofilm that may be left behind wiper 11, and thinner bristles 131 may scrub and help release or loosen contaminants lodged in the surface imperfections formed in the lumen's wall(s) to facilitate subsequent cleaning or decontamination of lumen L.

In various embodiments of scrubbers described herein that include bristle brushes, suitable bristle thicknesses (e.g., diameters) and materials may be selected based on the specific application and anticipated cleaning needs. For example, the bristles may be made from nylon or other suitable material(s). The bristles of the scrubbers described herein may be of uniform or non-uniform thicknesses. Examples of suitable bristle thicknesses (e.g., diameters) in some applications may be about 0.001″ (0.03 mm), about 0.002″ (0.05 mm), about 0.003″ (0.08 mm), about 0.004″ (0.10 mm), about 0.005″ (0.13 mm), about 0.006″ (0.15 mm), about 0.007″ (0.18 mm), about 0.008″ (0.20 mm), about 0.009″ (0.23 mm) or about 0.010″ (0.25 mm) for example.

FIG. 6 is a side view of another exemplary lumen cleaning tool 300 (referred hereinafter as “tool 300”). Tool 300 may have components previously described above in relation to tool 100. Like elements are identified using like reference numerals. In some embodiments, tool 300 may include a plurality of scrubbers 12 and a plurality of wipers 11. Scrubbers 12 and wipers 11 may be alternatingly disposed one after the other along core 10. Accordingly, as tool 300 is propelled through lumen L, the arrangement of scrubbers 12 and wipers 11 may perform alternating scrubbing and wiping operations on the wall(s) defining lumen L.

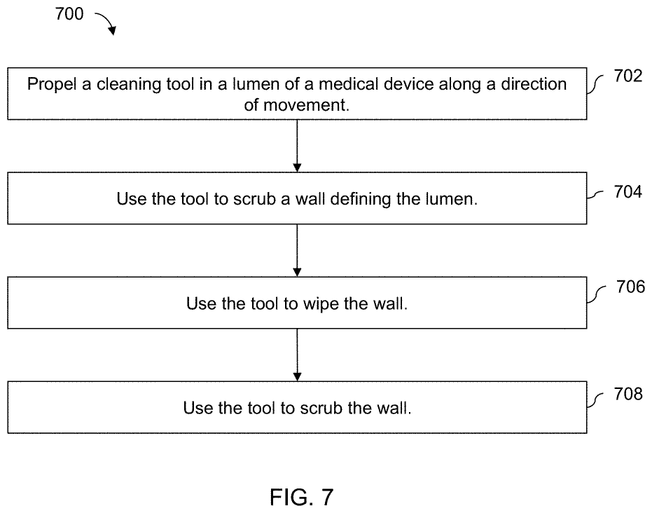

FIG. 7 is a flow diagram of method 700 for cleaning lumen L of a medical device. Method 700 may be performed with lumen cleaning tools described herein or with other lumen cleaning tools. Aspects or steps associated with lumen cleaning tools described herein may be incorporated into method 700. Method 700 may include:

propelling tool 100, 200 or 300 in lumen L along direction D (block 702); and

while propelling tool 100, 200 or 300 in lumen L along direction D, using tool 100, 200 or 300 to, in a single pass of tool 100, 200 or 300 in lumen L,

scrub a wall defining lumen L (block 704);

after scrubbing the wall, wipe the wall (block 706); and

after wiping the wall, scrub the wall (block 708).

In various embodiments of method 700, tool 100, 200 or 300 may be passed a single time or multiple times through lumen L to achieve the desired cleaning level. However, the configurations of tool 100, 200 or 300 may permit the actions of initial scrubbing, wiping and subsequent scrubbing to be performed in a single pass of tool 100, 200 or 300 through lumen L. The scrubbing-wiping-scrubbing sequence of operations carried out in a single pass may be achieved by way of wiper 11 being axially disposed between two scrubbers 12. Propelling of tool 100, 200 or 300 may be performed manually and/or may be performed using a pressure differential inside of lumen L as explained further below.

After having cleaned lumen L using tool 100, 200 or 300, lumen L may be decontaminated using one or more subsequent steps such as rinsing/flushing with a detergent solution.

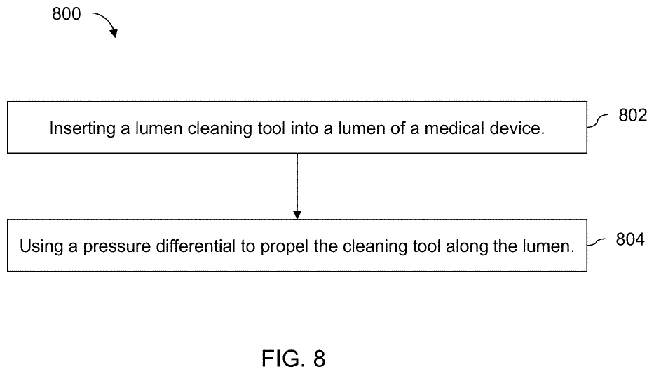

FIG. 8 is a flow diagram of method 800 of propelling a lumen cleaning tool along lumen L of a medical device using a pressure differential. Method 800 may be performed with lumen cleaning tools described herein or with other lumen cleaning tools. Aspects or steps associated with lumen cleaning tools described herein may be incorporated into method 800. Aspects of method 800 may be incorporated into method 700. Method 800 may be used in conjunction with lumen cleaning tools that include one or more scrubbers 12 and/or one or more wipers 11. Method 800 may include:

inserting tool 100, 200 or 300 into lumen L (see block 802); and

when tool 100, 200 or 300 is disposed inside lumen L, using a pressure differential inside lumen L to propel tool 100, 200 or 300 along lumen L.

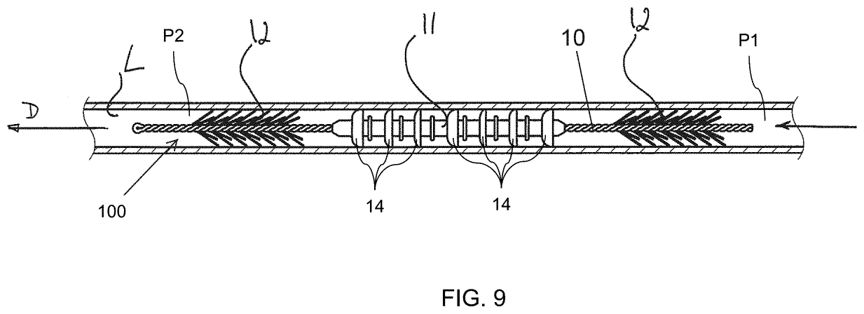

FIG. 9 is a side view of tool 100 disposed inside lumen L of a medical device and propelled inside lumen L using a pressure differential in accordance with method 800. As wiper 11 maintains a close contact with the wall(s) of lumen L, wiper 11 may act as a piston such that when a positive or negative pressure is applied at one end of the lumen L, tool 100 may be propelled along lumen L. In other words, disc-shaped squeegees 14 may provide a substantially complete circumferential seal inside lumen L so that a sufficient pressure differential (e.g., ΔP=P1−P2) axially across wiper 11 may urge and propel tool 100 along lumen L.

In reference to FIG. 9, a sufficient difference between pressure P1 and pressure P2 (i.e., P1>P2) may cause movement of tool 100 along lumen L. For example, the application of a positive pressure P1 behind wiper 11 (e.g., using a source of pressurized air or water) may be used to push tool 100 along lumen L in direction D. Instead or in addition, the application of a negative pressure P2 in front of wiper 11 (e.g., using a vacuum pump) may be used to pull tool 100 along lumen L in direction D.

The pressure differential may be applied by way of a pressurized fluid (e.g., gas and/or liquid) including air and/or water. For example, water pressurized to about 40 psi (276 kPa) may be used to apply a pressure differential along lumen L in order to propel tool 100 in the direction D. In some situations, the use of the pressure differential to propel tool 100 along lumen L may be less time consuming and easier than manually pulling or pushing tool 100 through lumen L. The use of a pressurized fluid to propel tool 100 may also provide additional rinsing of lumen L and may promote further removal of contaminants from lumen L.

The above description is meant to be exemplary only, and one skilled in the relevant arts will recognize that changes may be made to the embodiments described without departing from the scope of the invention disclosed. The present disclosure may be embodied in other specific forms without departing from the subject matter of the claims. The present disclosure is intended to cover and embrace all suitable changes in technology. Modifications which fall within the scope of the present invention will be apparent to those skilled in the art, in light of a review of this disclosure, and such modifications are intended to fall within the appended claims. Also, the scope of the claims should not be limited by the preferred embodiments set forth in the examples, but should be given the broadest interpretation consistent with the description as a whole.

What is claimed is:

1. A lumen cleaning tool for cleaning a lumen of a medical device, the lumen cleaning tool comprising:
an elongated core having a longitudinal axis;
a first bristle brush attached to the elongated core;
a second bristle brush attached to the elongated core, the first and second bristle brushes being axially spaced apart along the longitudinal axis of the elongated core; and
one or more disc-shaped squeegees attached to the elongated core and disposed axially between the first and second bristle brushes.

2. The lumen cleaning tool of claim 1, wherein:
the first bristle brush is disposed in front of the one or more disc-shaped squeegees and the second brush is disposed behind the one or more disc-shaped squeegees relative to a direction of movement of the lumen cleaning tool during use; and
the first bristle brush has first bristles of a first thickness;
the second bristle brush has second bristles of a second thickness;
the second thickness of the second bristles is smaller than the first thickness of the first bristles.

3. The lumen cleaning tool of claim 1, wherein the second bristle brush includes bristles of different thicknesses.

4. A lumen cleaning tool for cleaning a lumen of a medical device, the lumen cleaning tool comprising:
an elongated core;
two scrubbers attached to the elongated core and spaced apart along the elongated core; and
a wiper attached to the elongated core and disposed between the two scrubbers.

5. The lumen cleaning tool of claim 4, wherein the two scrubbers each includes a bristle brush.

6. The lumen cleaning tool of claim 4, wherein the wiper includes one or more squeegees.

7. The lumen cleaning tool of claim 4, wherein:
a first of the two scrubbers includes a first brush including first bristles of a first thickness; and
a second of the two scrubbers includes a second brush including second bristles of a second thickness different from the first thickness.

8. The lumen cleaning tool of claim 7, wherein:
the first brush is disposed in front of the wiper and the second brush is disposed behind the wiper relative to a direction of movement of the lumen cleaning tool during use; and
the second thickness of the second bristles is smaller than the first thickness of the first bristles.

9. The lumen cleaning tool of claim 4, wherein:
a first of the two scrubbers is disposed in front of the wiper and a second of the two scrubbers is disposed behind the wiper relative to a direction of movement of the lumen cleaning tool during use; and
the second scrubber includes a brush having bristles of different thicknesses.

10. The lumen cleaning tool of claim 4, wherein:
the two scrubbers are first and second scrubbers;
the lumen cleaning tool includes a third scrubber attached to the elongated core and spaced apart from the second scrubber;
the wiper is a first wiper;
the lumen cleaning tool includes a second wiper disposed between the second and third scrubbers; and
the second scrubber is disposed between the first and second wipers.

11. The lumen cleaning tool of claim 4, wherein:
the two scrubbers are two of a plurality of scrubbers attached to the elongated core;
the wiper is one of a plurality of wipers attached to the elongated core; and
the scrubbers and the wipers are alternatingly disposed one after the other along the elongated core.

12. A method of cleaning a lumen of a medical device, the method comprising:
propelling a cleaning tool in the lumen along a direction of movement; and
while propelling the cleaning tool in the lumen along the direction of movement, using the cleaning tool to, in a single pass of the cleaning tool in the lumen:
scrub a wall defining the lumen;
after scrubbing the wall, wipe the wall; and
after wiping the wall, scrub the wall.

13. The method of claim 12, comprising propelling the cleaning tool using a pressure differential inside the lumen.

14. The method of claim 13, comprising using a positive pressure in the lumen to push the cleaning tool along the lumen.

15. The method of claim 13, comprising using a negative pressure in the lumen to pull the cleaning tool along the lumen.

16. The method of claim 12, wherein the cleaning tool includes a first scrubber, a second scrubber, and a wiper disposed between the first and second scrubbers.

17. The method of claim 12, comprising decontaminating the lumen after propelling the cleaning tool in the lumen.

18. A method of propelling a lumen cleaning tool including a scrubber and a wiper along a lumen of a medical device, the method comprising:
when the lumen cleaning tool is disposed inside the lumen, using a pressure differential inside the lumen to propel the lumen cleaning tool along the lumen.

19. The method of claim 18, comprising using a positive pressure behind the wiper to push the lumen cleaning tool along the lumen.

20. The method of claim 18, comprising using a negative pressure in front of the wiper to pull the lumen cleaning tool along the lumen.
